(19) United States
(12) Patent Application Publication
(10) Pub. No.: US 2025/0315682 A1
(43) Pub. Date: Oct. 9, 2025

(54) Systems, Methods, And Devices For Customizable Computing Platforms

(71) Applicant: Salesforce, Inc., San Francisco, CA (US)

(72) Inventors: Claudia Brak, Carmel Valley, CA (US); Guivia Moreira, Atlanta, GA (US); Salil Pitkar, Foster City, CA (US); Tim Stokes, Los Angeles, CA (US); Udai Arora, San Francisco, CA (US); Nakul Pandey, Antioch, CA (US)

(73) Assignee: Salesforce, Inc., San Francisco, CA (US)

(21) Appl. No.: 18/628,089

(22) Filed: Apr. 5, 2024

(51) Int. Cl. G06N 3/0895; G16H 40/20

(52) U.S. Cl. CPC G06N 3/0895; G16H 40/20

(57) ABSTRACT

Systems, methods, and devices disclosed herein provide integration of on-demand applications with generative artificial intelligence platforms. For example, a computing platform may be implemented using a server system, where the computing platform is configurable to cause receiving application data from an on-demand application hosted by the computing platform, generating a data model based, at least in part, on the application data, the data model being a calendar data structure associated with a calendaring application, and generating, using an application model, additional application data, the application model being a machine learning model. The computing platform may be further configurable to cause updating the calendar data structure of the data model based, at least in part, on the additional application data, wherein the updating is performed, at least in part, via a plurality of custom data fields of a plurality of custom data objects.

### FIELD OF TECHNOLOGY

This patent application relates generally to computing platforms, and more specifically to improving implementation of operations within such computing platforms.

### BACKGROUND

“Cloud computing” services provide shared resources, applications, and information to computers and other devices upon request. In cloud computing environments, services can be provided by one or more servers accessible over the Internet rather than installing software locally on in-house computer systems. Users can interact with cloud computing services to undertake a wide range of tasks. Such cloud computing environments may be used to host distributed applications that may be used to support various distributed services provided to users. Conventional techniques for providing such services remain limited because they are not able to efficiently and effectively schedule and perform operations for distributed applications, or support custom data objects underlying such operations.

### BRIEF DESCRIPTION OF THE DRAWINGS

The included drawings are for illustrative purposes and serve only to provide examples of possible structures and operations for the disclosed inventive systems, apparatus, methods, and computer program products for implementing computing platforms. These drawings in no way limit any changes in form and detail that may be made by one skilled in the art without departing from the spirit and scope of the disclosed implementations.

FIG. 1 illustrates an example of a system for implementing a computing platform, configured in accordance with some implementations.

FIG. 2 illustrates an example of another system for implementing a computing platform, configured in accordance with some implementations.

FIG. 3 illustrates an example of a system for implementing an application model associated with a computing platform, configured in accordance with some implementations.

FIG. 4 illustrates a flow chart of an example of a method for generating application data, performed in accordance with some implementations.

FIG. 5 illustrates a flow chart of an additional example of a method for generating application data, performed in accordance with some implementations.

FIG. 6 illustrates a flow chart of an example of a method for generating an application model, performed in accordance with some implementations.

FIG. 7 illustrates a flow chart of an example of a method for generating application data, performed in accordance with some implementations.

FIG. 8 illustrates a flow chart of an additional example of a method for generating application data, performed in accordance with some implementations.

FIG. 9 illustrates a flow chart of another example of a method for generating application data, performed in accordance with some implementations.

FIG. 10 illustrates a block diagram of an example of an environment 910 that includes an on-demand database service configured in accordance with some implementations.

FIG. 11A illustrates a system diagram of an example of architectural components of an on-demand database service environment 1000, configured in accordance with some implementations.

FIG. 11B illustrates a system diagram further illustrating an example of architectural components of an on-demand database service environment, in accordance with some implementations.

FIG. 12 illustrates one example of a computing device.

### DETAILED DESCRIPTION

Implementations disclosed herein provide custom data objects capable of being implemented within the context of one or more on-demand applications to support integration with other on-demand applications as well as generative artificial intelligence platforms. As will be discussed in greater detail below, such custom data objects may be included within a calendar data structure of a calendaring application. Moreover, the custom data objects may include custom fields that may be configured to support integration with other on-demand applications such that events scheduled and actions taken within the calendaring application may be used to invoke and manage operations and actions in the other on-demand applications. Moreover, the custom data objects may also support the ability to augment the calendar data structure with application data provided by a machine learning model that may, for example, be implemented in a generative artificial intelligence platform such as Einstein provided by Salesforce.com®. In this way, the calendaring application may be configured to manage operations associated with other hosted applications, and also leverage input provided by the machine learning model implemented in a generative artificial intelligence platform.

In one example, a calendar data structure may be implemented in association with an on-demand application that is used to implement one or more healthcare process flows. Such process flows may be hosted by the on-demand application, and may be used to manage a sequence of operations included in a healthcare process, such as a series of appointments and follow-ups. Accordingly, an instance of the on-demand application may include one or more healthcare process flows which include sequences of events related to a patient and their corresponding treatment. There may also be associated data, such as survey information, notes, and other relevant information.

As will be discussed in greater detail below, custom data objects may be included within a calendar data structure of a calendaring application that enable integration of the healthcare process flows with the calendar data structure. More specifically, data events within the healthcare process flows may be mapped to custom event objects within the calendar data structure, and the calendar data structure may be used to manage aspects of the healthcare process flows, such as scheduling of appointments and sending of notifications and messages. Moreover, an application model, that may be a machine learning model, may be used to generate recommended events and appointments integrated within the calendar data structure and corresponding to the healthcare process flows. In this way, a generative machine learning model may augment event data and process flow data to improve the efficiency of generation and implementation of such healthcare process flows.

In another example, a calendar data structure may be implemented in associated with an on-demand application that is used to implement one or more advertisement campaigns. Accordingly, an instance of the on-demand application may include one or more advertisement campaigns which include advertisement objects provided to users. Moreover, there may be performance data, such as engagement metrics, actions taken and conversion rates, budget and financial information, as well as associated data such as contacts and leads.

In various implementations, custom data objects may be included within a calendar data structure that enable integration of the advertisement campaign with the calendar data structure. More specifically, data events within the advertisement campaigns may be mapped to custom event objects within the calendar data structure, and the calendar data structure may be used to manage aspects of the advertisement campaign, such as publication events and advertisement campaign scheduling. Moreover, an application model, that may be a machine learning model, may be used to generate recommended events integrated within the calendar data structure and corresponding to the advertisement campaign. In this way, a generative machine learning model may augment event data and advertisement campaign data to improve the efficiency of generation and implementation of such advertisement campaigns.

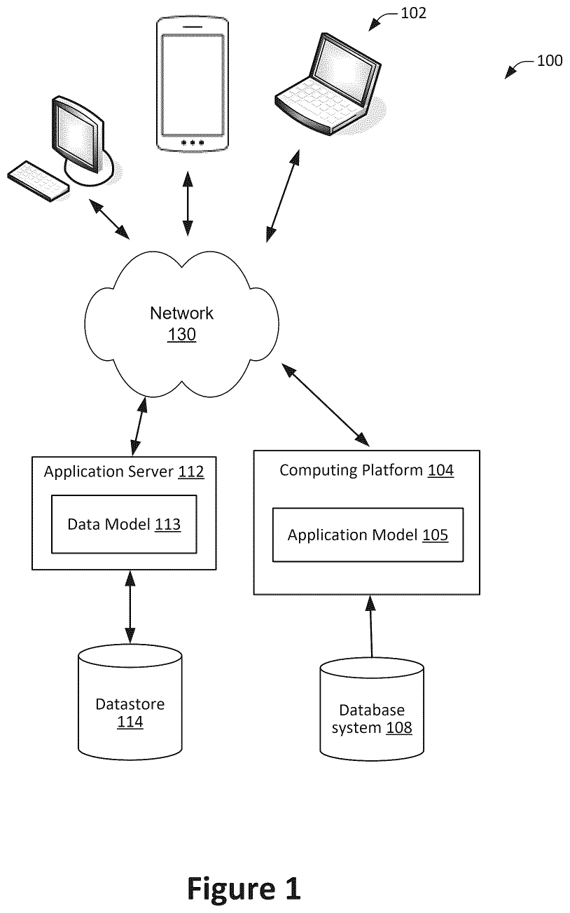

FIG. 1 illustrates an example of a system for implementing a computing platform, configured in accordance with some implementations. As will be discussed in greater detail below, components of a computing platform may be configured to communicate with an application server that may host a data model, and custom data objects may enable integration between the two. More specifically, the custom data objects may support custom data fields and/or custom Application Program Interfaces (APIs) that facilitate integration with functionalities of the computing platform, such an artificial intelligence platform, with the data model.

In various implementations, system 100 includes various client machines, which may also be referred to herein as user devices, such as client machine 102. In various implementations, client machine 102 is a computing device accessible by a user. For example, client machine 102 may be a desktop computer, a laptop computer, a mobile computing device such as a smartphone, or any other suitable computing device. Accordingly, client machine 102 includes one or more input and display devices, and is communicatively coupled to communications network 130, such as the internet. In various implementations, client machine 102 is configured to execute one or more applications that may utilize a user interface. Accordingly, a user may provide one or more inputs via client machine 102. In various implementations, a user interface may be used to present a webpage to the user. Accordingly, the user interface may utilize a web browser executed on client machine 102.

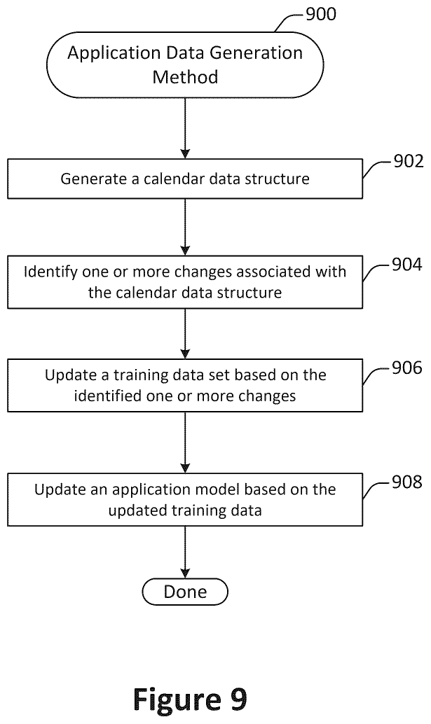

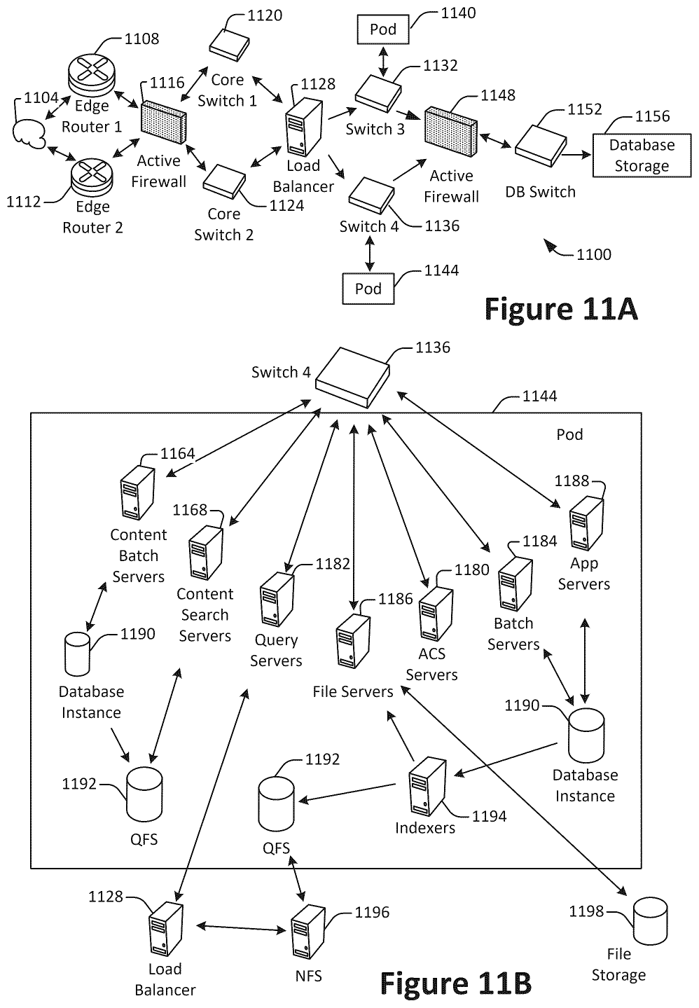

System 100 further includes application server 112. In some implementations, application server 112 may be implemented as discussed in greater detail below with reference to FIG. 9 and FIG. 11. In some implementations, application server 112 is configured to generate and serve webpages that may be viewed by a user via one or more devices, such as client machine 102. Accordingly, in some implementations, application server 112 includes a web server.

In various implementations, application server 112 further includes data model 113. As will be discussed in greater detail below, application server 112 may be configured to host on-demand applications that have underlying data models defining relationships and dependencies between data objects, and also defining parameters of general data objects. In some implementations, an on-demand application may be configured to support one or more operations, such as calendaring, advertisement campaign management, and/or workflow management. Thus, as will also be discussed in greater detail below, the data model may be an on-demand calendar application capable of providing multiple calendar views of events and appointments for a user as well as an organization. In various implementations, the on-demand application may provide a user interface through which a user may generate and manage data for an organization, and such data objects may be linked to data stored in multi-tenant customer relationship management (CRM) database, such as database system 108. In various implementations, such application data as well as other associated information may be stored in a datastore, such as datastore 114.

As will be discussed in greater detail below, data model 113 is configured to include custom data objects that may include content data as well as custom data fields and/or custom APIs configured to enable communications and integration with other on-demand applications and components of system 100, such as computing platform 104 discussed in greater detail below. Accordingly, the custom data objects may be configured to implement custom data fields and/or custom APIs to allow integration of calendaring functionality with those other components. Moreover, as will also be discussed in greater detail below, a machine learning model may be used to generate data for data model 113 via the custom data objects. In this way, machine learning capabilities may be leveraged within the calendaring application.

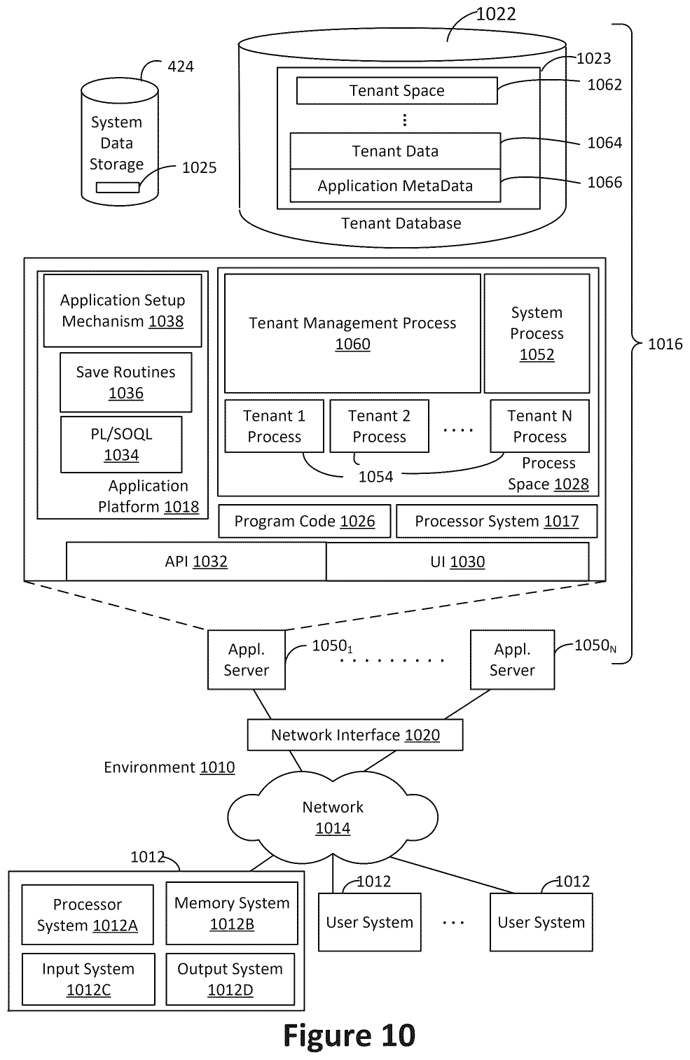

System 100 additionally includes computing platform 104. As shown in FIG. 1, computing platform may also be coupled to database system 108. As discussed in greater detail below with reference to FIG. 9, FIG. 10, and FIG. 11, computing platform 104 is configured to host one or more distributed on-demand applications. For example, computing platform 104 may be configured to host one or more on-demand applications provided by Salesforce.com®, such as the Salesforce Einstein platform. Accordingly, computing platform 104 may be configured to configure and implement application model 105 which may include one or more machine learning models configured to provide generative artificial intelligence. In various implementations, computing platform 104 may also include an interface configured to handle function calls, also referred to herein as server calls, generated by application server 112. The interface may be implemented using components of a database system, such as an API

As similarly discussed above, computing platform 104 is coupled to database system 108, which is configured to provide data storage utilized by computing platform 104. In various implementations, database system 108 includes system data storage and a tenant database, as discussed in greater detail below with reference to FIG. 9. In various implementations, computing platform 104 is also coupled to communications network 130, and is communicatively coupled to application server 112 and client machine 102.

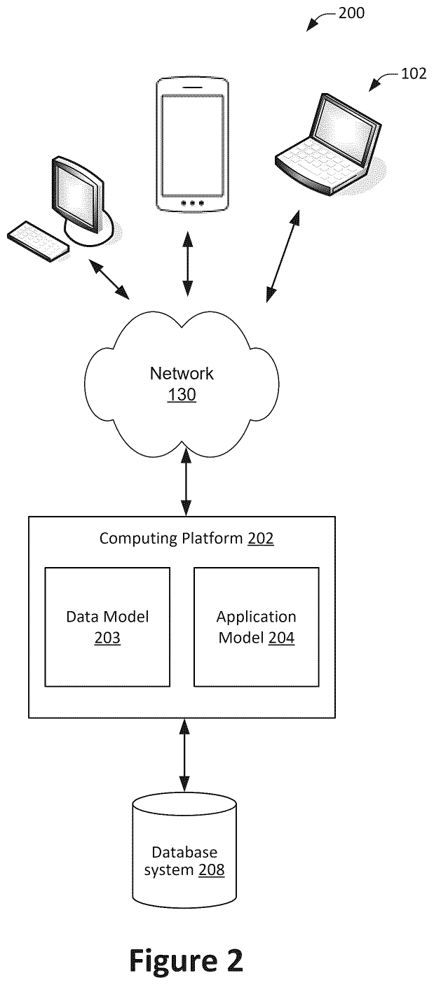

FIG. 2 illustrates an example of another system for implementing a computing platform, configured in accordance with some implementations. As shown in FIG. 2, system 200 may also include client machine 102 and network 103. Moreover, system 200 may also include computing platform 202 and database system 208 configured to store data associated with computing platform 202. In various implementations, computing platform 202 is configured to implement on-demand applications provided by Salesforce.com®, such as the Salesforce Einstein platform, as similarly discussed above. Accordingly, computing platform 202 may implement application model 203. Moreover, computing platform 202 may also be configured to implement on-demand applications using data models, such as data model 204. As similarly discussed above, data model 204 may be configured to support one or more operations, such as calendaring, advertisement campaign management, and/or workflow management. More specifically, data model 204 may be configured to include custom data objects that may include content data as well as custom data fields configured to enable communications and integration with other on-demand applications and components of system 200. Thus, as shown in FIG. 2, data model 204 and one or more machine learning models, such as application model 203, may be implemented within computing platform 202, and communication with an application server is not utilized.

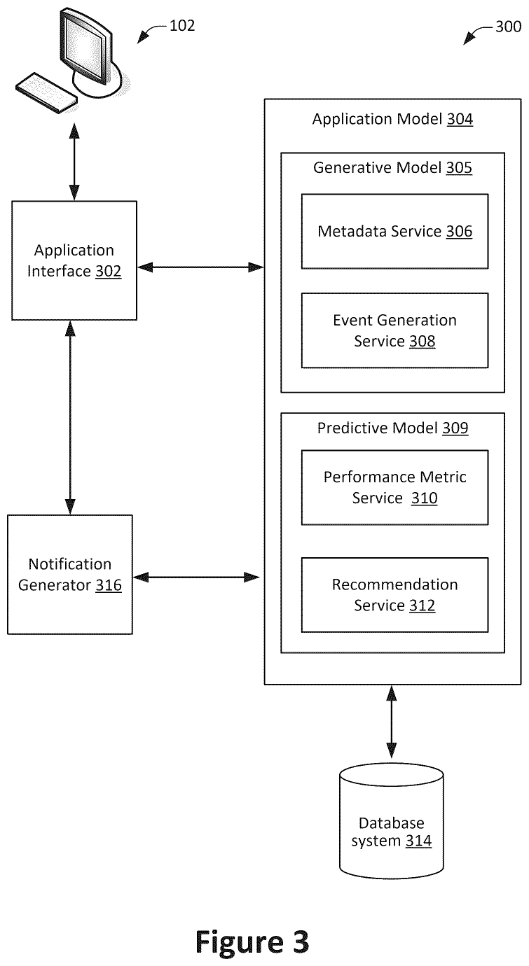

FIG. 3 illustrates an example of a system for implementing an application model associated with a computing platform, configured in accordance with some implementations. As discussed above, an application model may include one or more machine learning models configured to augment data and services associated with an on-demand application, such as a calendaring application. As will be discussed in greater detail below, a system, such as system 300, may be implemented to provide generative and predictive machine learning services for such an on-demand application.

In various implementations, system 300 includes client machine 102. As similarly discussed above, client machine 102 is a computing device accessible by a user. For example, client machine 102 may be a desktop computer, a laptop computer, a mobile computing device such as a smartphone, or any other suitable computing device. Accordingly, client machine 102 includes one or more input and display devices that may be used by a user to access an on-demand application provided by an entity, such as Salesforce.com. Client machine 102 may be communicatively coupled to application interface 302 which may be configured to provide web services for a computing platform used to implement the on-demand application. Accordingly, application interface 302 is configured to manage communications between client machine 102 and other components of the computing platform.

System 300 further includes application model 304 which includes one or more machine learning models, such as generative model 305 and predictive model 309. As discussed above, application model 304 may be implemented in a computing platform or in an application server, and may be implemented using servers and computing devices as discussed below with reference to FIGS. 10-12. More specifically, generative model 305 and predictive model 309 may each be implemented using a respective cluster of compute resources including processors and memory configured to support implementation of machine learning processing operations, such as neural networks and large language models (LLMs).

Accordingly, application model 304 may include generative model 305 which is an LLM configured to generate text for event objects and data fields of such event objects. The LLM may have been previously trained on historical scheduling and calendar data, and may generate text for event objects based on a received input. More specifically, generative model 305 may include metadata service 306 which may be a specific instance of an LLM configured to generate such text for data fields, and to generate an output in a JSON or CSV format provided to one or more other components of application model 304, such as predictive model 309 discussed in greater detail below. In one example, the output of metadata service 306 may be used to populate and augment feature fields and labels associated with existing event objects and event groups. Accordingly, metadata service 306 may be configured to enrich features used by other system components, such as predictive model 309.

In various implementations, predictive model 309 is a predictive machine learning model that may include one or more neural networks trained on previous historical data. For example, predictive model 309 may include one or more services, such as performance metric service 310 which may be a neural network trained on previous historical data including previous events and associated performance data that may include metrics such as landing page views, click throughs, conversion rates, and form submissions. In various implementations, performance metric service 310 may receive the output of metadata service 306 as well as current calendar data structure information, and may generate predictions for various performance metrics based on the current calendar data structure information which may include a current configuration of events. Accordingly, based on the previous training of the neural network, performance metric service 310 may estimate outcomes for various performance metrics, such as page views, click throughs, conversion rates, and form submissions.

In various implementations, predictive model 309 may also include recommendation service 312 which may be a separate instance of a neural network configured to modify parameters of a current configuration of events in the calendar data structure, and generate one or more recommendations based on such modifications. For example, recommendation service 312 may modify a date on which an event has been scheduled in accordance with a specified temporal range, such as plus or minus 5 days. Recommendation service 312 may also be a neural network trained on previous historical data including previous events and associated performance data. Accordingly, recommendation service 312 may generate estimates for each modification, and may select the configuration resulting in the best estimated performance metrics as a recommendation. Recommendation service 312 may generate an output having a JSON or CSV format, and may provide the output to one or more components of generative model 305, as similarly discussed below. It will be appreciated that generative model 305 and predictive model 309 may be iteratively updated and retrained as new data is stored in database system 314.

In various implementations, an output of performance metric service 310 may be provided to a component of generative model 305, such as event generation service 308. In one example, event generation service 308 may be a separate instance of an LLM configured to generate event objects based on the output of performance metric service 310. More specifically, performance metric service 310 may generate an output that may have a JSON or CSV format, and the output may include various estimations of outcomes for an existing configuration of events in a calendar data structure. In various implementations, event generation service 308 may receive the output from performance metric service 310, and may generate event objects that include representations of the estimated outcomes. Thus, event generation service 308 may be configured to provide a natural language representation of estimated outcomes within the format of the current configuration of events in the calendar data structure.

In some implementations, generation and augmentation of data by application model 304 is performed dynamically and responsive to an input provided by a user. In one example, a user may provide an input via application interface 302 while configuring a calendar data object. Accordingly, the user may be provided with a dynamic event form in which inputs provided to the event form trigger actions of generative model 305 and predictive model 309 dynamically. In some embodiments, the dynamic event form may include custom data fields and/or custom APIs that are configured to function calls to trigger invocation of application model 304. Accordingly, application model 304 may be configured to render components of a dynamic UI provided to the user and also augment content via custom data fields, and the user may modify parameters, such as event dates, publishing times, distribution lists, and communications channels, until the user is satisfied with the estimated outcome. The user may then choose an action, such as selecting a configuration events or an event group, to terminate the dynamic modification process. Accordingly, the user may accept recommended changes, and terminate the dynamic modification process.

In various implementations, system 300 additionally includes notification generator 316 which is configured to generate one or more messages based, at least in part, on an output of application model 304. For example, notification generator 316 may be configured to generate web-based messages, such as email messages, based on generated data objects, such as events. Accordingly, notification generator 316 may generate and transmit email messages associated with events and event groups generated by application model 304.

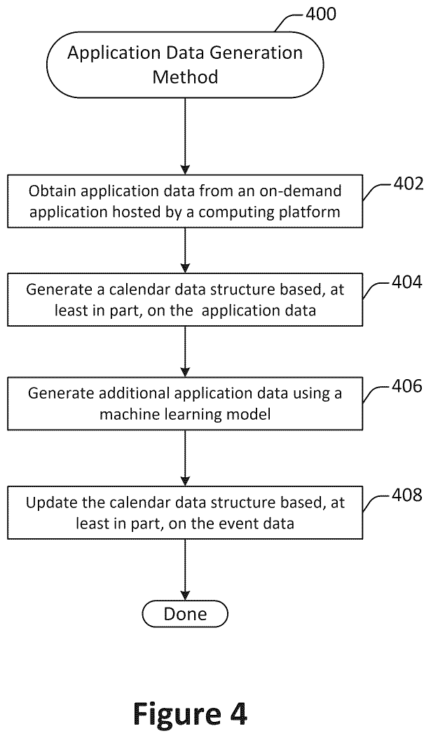

FIG. 4 illustrates a flow chart of an example of a method for generating application data, performed in accordance with some implementations. As similarly discussed above, application data may be data underlying an on-demand application provided by an entity, such as Salesforce.com. For example, such application data may include CRM data stored in a multitenant database system, advertisement campaign data, as well as data associated with various other on-demand services. In various implementations, a method, such as method 400, may be performed to generate a data model based on such application data, as well as augment the data model using one or more machine learning models.

Method 400 may perform operation 402 during which application data may be obtained from an on-demand application hosted by a computing platform. As discussed above, the on-demand application may be configured to support one or more on-demand services, such as the implementation of an advertisement campaign. Accordingly, the application data may include advertisement campaign data objects identifying advertisement campaigns, performance metrics, as well as one or more identifiers associated with entities within an organization. Moreover, the application data may further include associated CRM data.

Method 400 may perform operation 404 during which a data model may be generated based, at least in part, on the application data. In various implementations, the data model is a calendar data structure that is generated based, at least in part, on the application data. For example, the data model may be a calendar data structure configured to provide a calendar view of various events. During operation 404, the calendar data structure may be populated based on the retrieved application data. In one example, the calendar data structure may be populated based on advertisement campaign data such that events are generated within the calendar data structure corresponding to events within the advertisement campaign. Such population of the calendar data structure may be implemented via a mapping of campaign data object identifiers to event identifiers.

Method 400 may perform operation 406 during which additional application data may be generated using a machine learning model. In various implementations, the application data as well as the data model may be provided to a generative machine learning model, and the machine learning model may generate additional data based on the received input. For example, the machine learning model may generate additional events within the calendar data structure, or may make modifications and changes to existing events within the calendar data structure. In this way, the machine learning model may be used to supplement and modify the data model and its underlying application data.

Method 400 may perform operation 408 during which the data model may be updated based, at least in part, on event data. Accordingly, the additional data generated by the machine learning model may be integrated with the data model, and events within the data model may be updated accordingly. For example, the calendar data structure may be updated to include additional events generated by the machine learning model, and other events that may have identified dependencies may be updated as well. In this way, an data model, such as a calendar data structure, may be generated, populated, augmented, and updated.

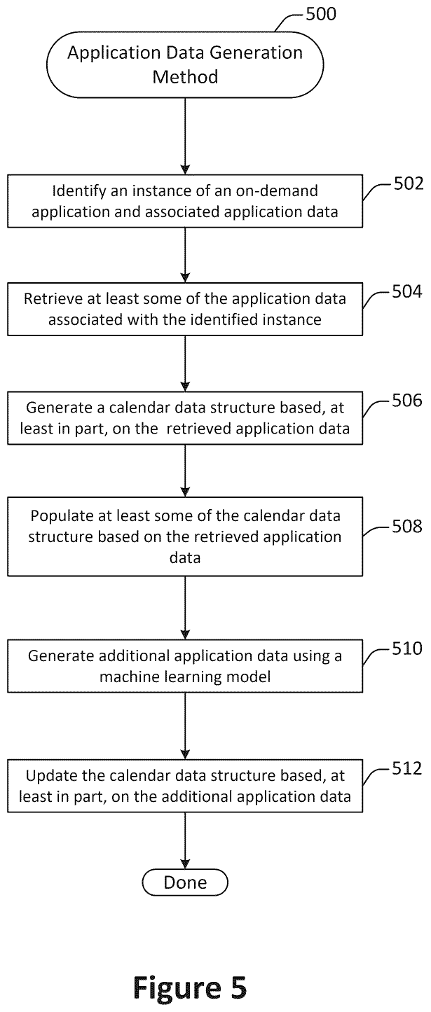

FIG. 5 illustrates a flow chart of an additional example of a method for generating application data, performed in accordance with some implementations. As similarly discussed above, application data may be data underlying an on-demand application provided by an entity, such as Salesforce.com. In various implementations, a method, such as method 500, may be performed to generate a calendar data structure that corresponds to such on-demand applications. Moreover, a machine learning model may be used to improve the generation and population of such a calendar data structure.

Method 500 may perform operation 502 during which an instance of an on-demand application and associated application data may be identified. In various implementations, the instance may be identified based on one or more identifiers associated with a user or an organization. For example, a user may have requested the generation of a calendar data structure, and the user may have an associated user profile associated with the on-demand application. Such a user profile may identify various associations, such as an associated organization, as well as a role within the organization. Moreover, the user profile as well as associated credential information may be mapped to a particular instance of the on-demand application. More specifically, the instance of the on-demand application for the user's organization may be identified.

Method 500 may perform operation 504 during which at least some of the application data associated with the identified instance may be retrieved. As similarly discussed above, the application data may include advertisement campaign data objects identifying advertisement campaigns, performance metrics, as well as one or more identifiers associated with entities within an organization. Moreover, the application data may further include associated CRM data. In various embodiments, a portion of the application data may be identified by the user, and may be retrieved. For example, the user may specify that application data for a particular advertisement campaign should be retrieved.

Method 500 may perform operation 506 during which a calendar data structure may be generated based, at least in part, on the retrieved application data. In various implementations, the calendar data structure may be generated based on a template as well as the retrieved application data. For example, the calendar data structure may be part of an on-demand calendaring application provided by an entity, such as Salesforce.com. Accordingly, a calendar data structure template may be used to provide an initial instance of the calendar data structure. Moreover, application data and user data may be used to populate portions of the initial instance of the calendar data structure with identifying information specific to the user and the organization as well as an initial set of calendar events that may be pulled from one or more specified data sources, such as a user's calendar.

Method 500 may perform operation 508 during which at least some of the calendar data structure may be populated based on the retrieved application data. As similarly discussed above, the calendar data structure may be populated based on additional portions of the retrieved application data. For example, the calendar data structure may be populated based on advertisement campaign data such that events are generated within the calendar data structure corresponding to events within the advertisement campaign. Such population of the calendar data structure may be implemented via a mapping of campaign data object identifiers to event identifiers.

Moreover, as will be discussed in greater detail below, event and data objects created within the calendar data structure may be custom data objects that are configured to enable interaction and integration with other on-demand applications. For example, event objects may have custom data fields that define data object associations, define user interface and view configurations, and may be used to generate and support custom data fields and/or APIs. Accordingly, during operation 508, such custom data fields may be generated and configured based on one or more configuration parameters that may be specified by, for example, a user.

Method 500 may perform operation 510 during which additional application data may be generated using a machine learning model. As similarly discussed above, the application data as well as the data model may be provided to a generative machine learning model, and the machine learning model may generate additional data based on the received input. For example, the machine learning model may generate additional events for the calendar data structure, or may identify modifications and changes to existing events within the calendar data structure.

In various implementations, the additional data may include configurations of event data that have been generated based on estimations of outcomes made based on previous events. For example, as will be discussed in greater detail below, previous advertisement campaigns may have been used to train the machine learning model, and the machine learning model may be configured to generate recommended campaigns that are configured to provide estimated outcomes based on a set of input parameters that may be inferred from the application data and/or received from a user.

For example, a timing of an event may be recommended by the machine learning model based on past performance data In this way, parameters of individual events as well as a configuration and distribution of an entire group of events may be generated by the machine learning model, and provided as a recommendation, and/or integrated with existing events included in application data. In one example, a type of on-demand application and past performance data associated with, for example, an on-demand workflow management application, may be used to generate a configuration and distribution of a group of events within a calendaring application for a particular workflow data object.

Method 500 may perform operation 512 during which the calendar data structure may be updated based, at least in part, on the additional application data. As similarly discussed above, the additional data generated by the machine learning model may be integrated with the data model, and events within the data model may be updated accordingly. For example, the calendar data structure may be updated to include additional events generated by the machine learning model, and other events that may have identified dependencies may be updated as well. Moreover, as will be discussed in greater detail below, the additional application data generated by the machine learning model may be integrated with events included in the calendar data structure via one or more custom data fields and/or APIs provided by custom data fields of the event objects.

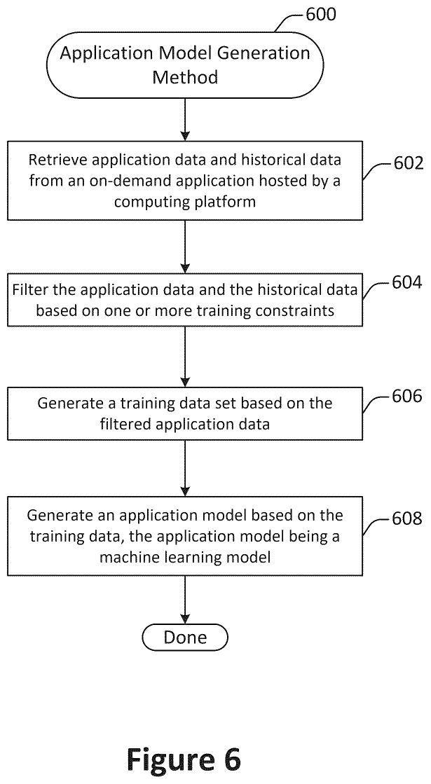

FIG. 6 illustrates a flow chart of an example of a method for generating an application model, performed in accordance with some implementations. As similarly discussed above, an application model may be used to generate data objects for a data model as well as populate the data model with the generated content. As will be discussed in greater detail below, a method, such as method 600 may be performed to configure and implement a machine learning model based, at least in part, on constraints which may be configuration parameters specified by an entity, such as a user or an organization.

Method 600 may perform operation 602 during which application data and historical data may be retrieved from an on-demand application hosted by a computing platform. In various implementations, the application data and historical data may include previous application data and user data that may be associated with a user and/or an organization. The application data and historical data may include information such as configuration data regarding an instance of the on-demand application, and may also include existing data models and associated performance data and metrics. For example, the existing data models may be previous advertisement campaigns or healthcare flows, and the performance data may include metrics, such as engagement metrics that represent outcomes of such previous advertisement campaigns or healthcare flows. Such data may be stored in a multitenant database, and may be retrieved from the multitenant database during operation 602. In various implementations, the stored data may also be stored and retrieved in accordance with permissions settings and data anonymization policies. Accordingly, the data may be filtered based on one or more permissions settings, and may also be anonymized to remove identifying information.

In some implementations, the application data and historical data may include performance metrics and success criteria defined for previous iterations of the on-demand application. For example, historical data may include performance metrics that may also have associated data dimensions, such as event type and event group. The performance metrics may include observed interactions and engagement such as number of views, a number of through clicks, a number of conversions and/or transactions, a number of non-conversions. Performance metrics may also include additional success criteria, such as positive health outcomes and positive surveys being returned. Performance metrics may also include actual durations of appointments based on type, staff illnesses rates during certain times of the year, injury and illness rates of patients during certain periods of time, cancellation rates during certain periods of time. Such performance metrics may also be stored at a group level in which success criteria are tracked for a group of events over the duration of the iteration of the on-demand application, which may include the implementation of an advertisement campaign or a healthcare flow.

Method 600 may perform operation 604 during which the application data and the historical data may be filtered based on one or more training constraints. In various embodiments, the training constraints may be defined by a user or other entity, such as an administrator. The training constraints may be configured to apply filtering parameters to application data and historical data that is retrieved based on one or more data fields as well as metadata values. Accordingly, the application data and historical data may be filtered based on dimensions such as a timestamp, one or more identifiers identifying a selected dimension or data object, as well as any other suitable data field. For example, a user may select a particular advertisement campaign or healthcare flow, and only use application data and historical data for that selected advertisement campaign or healthcare flow.

Method 600 may perform operation 606 during which a training data set may be generated based on the filtered application data. Accordingly, once the application data and historical data has been filtered, it may be integrated into a data object having a format being capable of ingestion by a machine learning model. The data object may be stored as a training data set.

Method 600 may perform operation 608 during which an application model may be generated based on the training data. In various implementations, the application model is a machine learning model that is trained based on the training data set. More specifically, machine learning models may be supervised machine learning models, and may be used to generate event data based on initial application data. Accordingly, such machine learning models may be generated and implemented using a learning phase and an inference phase. In some embodiments, the machine learning models may be neural networks. Accordingly, the machine learning model may be trained based on the previous application data and historical data such that the resulting trained model is configured to generate event data based on an initial input inferred from current application data.

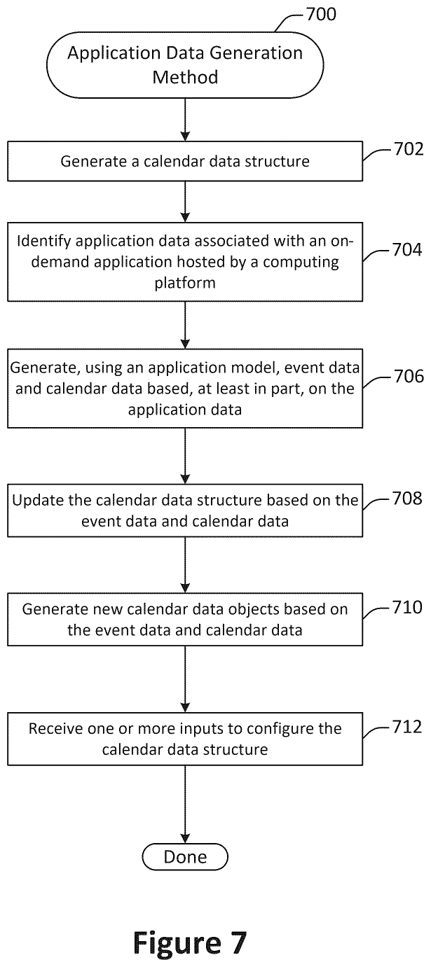

FIG. 7 illustrates a flow chart of an example of a method for generating application data, performed in accordance with some implementations. As discussed above an application model may be used to populate a data model, and such population may occur via the use of custom data fields of data objects within the data model. Accordingly, a method, such as method 700, may be performed to use such an application model to generate content for and integrate such content within a data model, which may be a calendar data structure.

Method 700 may perform operation 702 during which a calendar data structure may be generated. As similarly discussed above, the calendar data structure may be generated based on a template as well as the retrieved application data. For example, the calendar data structure may be part of an on-demand calendaring application provided by an entity, such as Salesforce.com. Accordingly, a calendar data structure template may provide an initial instance of the calendar data structure. Moreover, application data and user data may be used to populate portions of the initial instance of the calendar data structure by identifying information specific to the user and the organization as well as an initial set of calendar events.

Method 700 may perform operation 704 during which application data associated with an on-demand application hosted by a computing platform may be identified. In various implementations, the application data may include current application data for particular instances of on-demand applications. For example, such application data may identify currently active advertisement campaigns as well as associated advertisement campaign data. In another example, such application data may identify currently active healthcare flows associated with healthcare services and existing events associated with such healthcare flows.

Method 700 may perform operation 706 during which event data and calendar data may be generated based, at least in part, on the application data. In various implementations, the event data and calendar data may be generated using an application model. As discussed above, the application model may be a machine learning model configured to generate the event data and calendar data based on the identified application data. As will be discussed in greater detail below, the event data generated by the application model may include events and groups of events that include custom event objects having custom data fields. In some implementations the output generated by the application model may be packaged as a recommendation, and may be presented to a user via a user interface screen as a recommendation.

Moreover, the application model may also generate custom views associated with such event data and event groups. For example, a user interface may be generated that displays user interface icons for packages including groups of events that may be generated. In some embodiments, the user interface may be configured to receive an input from a user via a client device, and the input may be used to select a group of events to be generated. In this way, multiple possible sets of event data and calendar data may be presented to a user, and the user may provide an input to make such a selection.

Method 700 may perform operation 708 during which the calendar data structure may be updated based on the event data and the calendar data. Accordingly, the calendar data structure may be populated with the event data and calendar data generated during operation 706. As discussed above, the calendar data structure may be populated based on one or more selections made by a user. Moreover, one or more updates may be applied to existing event objects if there are existing events within the calendar data structure. In one example, standard event objects may be converted to custom event objects and be configured to have custom data fields.

Method 700 may perform operation 710 during which new calendar data objects may be generated based on the event data and the calendar data. In some implementations, a user may additionally provide an input that causes the generation of additional event objects. In this way, the user may also manually configure calendar data structure by making one or more modifications. Moreover, the calendar data structure may also be updated based on one or more identified dependencies between objects. For example, if a created event requires additional subsequent events, those additional events may be generated and populated as well.

Method 700 may perform operation 712 during which one or more inputs may be received to configure the calendar data structure. In various implementations, one or more additional inputs may be received from a user to configure a view associated with the calendar data structure as well as one or more permissions and associations with other entities within the user's organization. In this way, the user may configure how the calendar data structure is presented within the calendaring on-demand application, as well as which entities have what level of access.

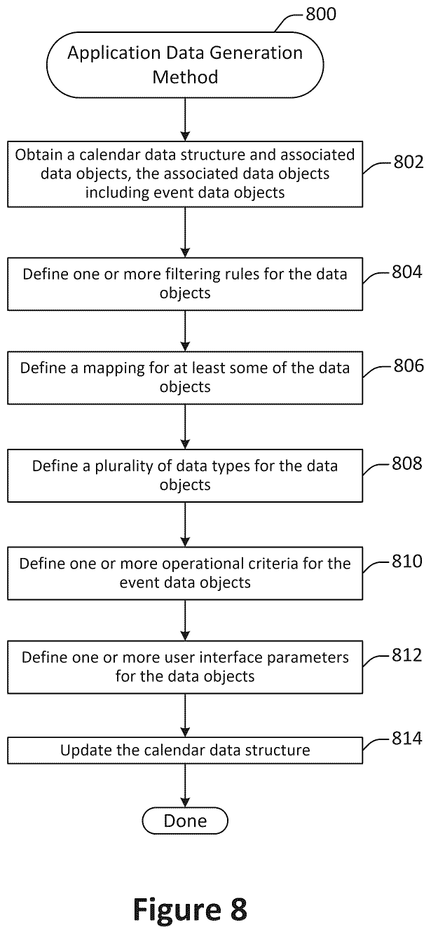

FIG. 8 illustrates a flow chart of an additional example of a method for generating application data, performed in accordance with some implementations. As discussed above, calendar data structures may be configured to include custom event objects configured to support custom data fields and/or APIs that enable interaction and integration with other applications in an on-demand environment. Accordingly, a method, such as method 800, may be performed to configure custom event objects to support such custom data fields and/or APIs.

Method 800 may perform operation 802 during which a calendar data structure and associated data objects may be obtained. In some implementations, the associated data objects may include event data objects as well as group data objects and task data objects. The calendar data structure may be stored in a multitenant database and may be retrieved in response to a request. In one example, the request may be received from a user in response to a determination that data objects should be configured. If no calendar data structure exists, one may be generated as discussed above.

In various implementations, the data objects may each have a set of data fields configured to store data relevant to its associated data type. For example, a custom event data object may have data fields identifying an action, a status, a workspace, a channel, and/or any other suitable parameter associated with the on-demand environment. As will be discussed in greater detail below, the data fields associated with a data object may be determined based on a defined data type of that data object.

Method 800 may perform operation 804 during which one or more filtering rules may be defined for the data objects. In various implementations, the filtering rules may define one or more properties associated with a data object, such as an event or an event group, which may be used for filtering that may be applied within the context of the calendar data structure. For example, such filtering rules may be used to define when data objects, such as a custom event object, may be visible in which view of the calendar data structure. Accordingly, such filtering rules may define how such data objects are presented in different views, such as a daily view, a weekly view, and a monthly view. Such filtering rules may also be used during queries executed by APIs on the data objects. Accordingly, during operation 804, such filtering rules may be determined by a user, or may be determined by an application model.

Method 800 may perform operation 806 during which a mapping may be defined for at least some of the data objects. In various implementations, such mappings may define data dependencies with other data objects and also define syncing relationships. For example, custom data fields of a custom event object may be mapped to data fields of a different data object, such as a standard event object. In this example, modification or updates to the custom data fields may cause updates to the data fields of the standard event object. Accordingly, during operation 806, such relationships with other data objects may be identified and defined.

Method 800 may perform operation 808 during which a plurality of data types may be defined for the data objects. As discussed above, data objects may be assigned a data type, such as an event, a task, or a group. Each data type may have data structure defined by a designated set of data fields. Accordingly, if not already defined, during operation 808, such data types may be defined for data objects, and/or may be changed for data objects. Moreover, sub-types of data objects may also be defined. For example, an event object may have one of multiple different types of events, such as a creation event or a publication event. In some implementations, the data type may also affect a type of action associated with the data object. For example, for a publication event object, an action specified by the event may be taken at a specified date of publication. Thus, during operation 808, a sub-type may also be defined and configured based on an input received from a user or by an application model.

Method 800 may perform operation 810 during which one or more operational criteria may be defined for the data objects. In various implementations, the operational criteria may define custom actions and integrations that may be associated with a data object. For example, a custom event object may have an associated action underlying the event, such as a message or notification generation, webpage publication, advertisement publication, or other data object generation. The operational criteria may be configured to define the action. Moreover, the operational criteria may define one or more associations with other components of a computing platform. For example, the operational criteria may define function call made to invoke a process flow of a separate on-demand application. In this way, custom data fields of custom data objects may be used to manage invocation and execution of process flows of on-demand applications via the calendaring application and the calendar data structure.

Method 800 may perform operation 812 during which one or more user interface parameters may be defined for the data objects. In various implementations the user interface parameters may configure a presentation and view of the data objects based on a selected view of the calendar data structure. For example, a first set of data fields may be visible in a first view, and a second set of data fields may be visible in a second view. In this way, a data object may be presented differently based on a view that is selected. In one example, a custom event object may have a first presentation in an event details view, and may have a second presentation in an event setup flow view.

Method 800 may perform operation 814 during which the calendar data structure may be updated. Accordingly, the calendar data structure obtained during operation 802 may be updated based on the defined items discussed above such that the custom data objects included in the calendar data structure are updated to include and reflect such defined items. In this way, custom data objects, such as custom event objects as well as groups of custom event objects, may be configured and implemented.

FIG. 9 illustrates a flow chart of another example of a method for generating application data, performed in accordance with some implementations. As discussed above, application models may be used to generate event data for calendar data structures. As will be discussed in greater detail below, the application model may be updated based on additional iterations of applications as well as additional updates to application data. In this way, the application model may be periodically and/or dynamically retrained to Method 900 may perform operation 902 during which a calendar data structure may be generated. As similarly discussed above, the calendar data structure may be generated based on a template as well as the retrieved application data. Moreover, the application data and user data may be used to populate portions of the initial instance of the calendar data structure with identifying information specific to the user and the organization as well as an initial set of calendar events. In some implementations, a calendar data structure may have been previously generated. Accordingly, during operation 902, a stored calendar data structure may be retrieved from a storage location.

Method 900 may perform operation 904 during which one or more changes associated with the calendar data structure may be identified. In various implementations the changes may be changes to data values included in event objects as well as the creation and/or deletion of event objects. For example, such changes may include changes made by a user, and/or the addition of additional application data that includes, for example, results of an advertisement campaign or results of a healthcare process flow. Such changes may be identified based on a changelog, or a comparison with a previous instance of the calendar data structure.

Method 900 may perform operation 906 during which a training data set may be updated based on the identified one or more changes. Accordingly, the identified changes may be used to update existing data in a training data set. In one example, event object identifiers may be used to map identified changes to data objects within the training data, and the training data may be updated to reflect the most recent changes. Moreover, it will be appreciated that the training data may also be updated to include new data objects if new event objects have been identified.

Method 900 may perform operation 908 during which an application model may be updated based on the updated training data. Accordingly, the updated training data may be used to re-train the application model, and the updated application model may be stored for future use.

FIG. 10 shows a block diagram of an example of an environment 1010 that includes an on-demand database service configured in accordance with some implementations. Environment 1010 may include user systems 1012, network 1014, database system 1016, processor system 1017, application platform 1018, network interface 1020, tenant data storage 1022, tenant data 1023, system data storage 1024, system data 1025, program code 1026, process space 1028, User Interface (UI) 1030, Application Program Interface (API) 1032, PL/SOQL 1034, save routines 1036, application setup mechanism 1038, application servers 1050-1 through 1050-N, system process space 1052, tenant process spaces 1054, tenant management process space 1060, tenant storage space 1062, user storage 1064, and application metadata 1066. Some of such devices may be implemented using hardware or a combination of hardware and software and may be implemented on the same physical device or on different devices. Thus, terms such as “data processing apparatus,” “machine,” “server” and “device” as used herein are not limited to a single hardware device, but rather include any hardware and software configured to provide the described functionality.

An on-demand database service, implemented using system 1016, may be managed by a database service provider. Some services may store information from one or more tenants into tables of a common database image to form a multi-tenant database system (MTS). As used herein, each MTS could include one or more logically and/or physically connected servers distributed locally or across one or more geographic locations. Databases described herein may be implemented as single databases, distributed databases, collections of distributed databases, or any other suitable database system. A database image may include one or more database objects. A relational database management system (RDBMS) or a similar system may execute storage and retrieval of information against these objects.

In some implementations, the application platform 1018 may be a framework that allows the creation, management, and execution of applications in system 1016. Such applications may be developed by the database service provider or by users or third-party application developers accessing the service. Application platform 1018 includes an application setup mechanism 1038 that supports application developers' creation and management of applications, which may be saved as metadata into tenant data storage 1022 by save routines 1036 for execution by subscribers as one or more tenant process spaces 1054 managed by tenant management process 1060 for example. Invocations to such applications may be coded using PL/SOQL 1034 that provides a programming language style interface extension to API 1032. A detailed description of some PL/SOQL language implementations is discussed in commonly assigned U.S. Pat. No. 7,730,478, titled METHOD AND SYSTEM FOR ALLOWING ACCESS TO DEVELOPED APPLICATIONS VIA A MULTI-TENANT ON-DEMAND DATABASE SERVICE, by Craig Weissman, issued on Jun. 1, 2010, and hereby incorporated by reference in its entirety and for all purposes. Invocations to applications may be detected by one or more system processes. Such system processes may manage retrieval of application metadata 1066 for a subscriber making such an invocation. Such system processes may also manage execution of application metadata 1066 as an application in a virtual machine.

In some implementations, each application server 1050 may handle requests for any user associated with any organization. A load balancing function (e.g., an F5 Big-IP load balancer) may distribute requests to the application servers 1050 based on an algorithm such as least-connections, round robin, observed response time, etc. Each application server 1050 may be configured to communicate with tenant data storage 1022 and the tenant data 1023 therein, and system data storage 1024 and the system data 1025 therein to serve requests of user systems 1012. The tenant data 1023 may be divided into individual tenant storage spaces 1062, which can be either a physical arrangement and/or a logical arrangement of data. Within each tenant storage space 1062, user storage 1064 and application metadata 1066 may be similarly allocated for each user. For example, a copy of a user's most recently used (MRU) items might be stored to user storage 1064. Similarly, a copy of MRU items for an entire tenant organization may be stored to tenant storage space 1062. A UI 1030 provides a user interface and an API 1032 provides an application programming interface to system 1016 resident processes to users and/or developers at user systems 1012.

System 1016 may implement a web-based calendaring system. For example, in some implementations, system 1016 may include application servers configured to implement and execute calendaring software applications. The application servers may be configured to provide related data, code, forms, web pages and other information to and from user systems 1012. Additionally, the application servers may be configured to store information to, and retrieve information from a database system. Such information may include related data, objects, and/or Webpage content. With a multi-tenant system, data for multiple tenants may be stored in the same physical database object in tenant data storage 1022, however, tenant data may be arranged in the storage medium(s) of tenant data storage 1022 so that data of one tenant is kept logically separate from that of other tenants. In such a scheme, one tenant may not access another tenant's data, unless such data is expressly shared.

Several elements in the system shown in FIG. 10 include conventional, well-known elements that are explained only briefly here. For example, user system 1012 may include processor system 1012A, memory system 1012B, input system 1012C, and output system 1012D. A user system 1012 may be implemented as any computing device(s) or other data processing apparatus such as a mobile phone, laptop computer, tablet, desktop computer, or network of computing devices. User system 12 may run an internet browser allowing a user (e.g., a subscriber of an MTS) of user system 1012 to access, process and view information, pages and applications available from system 1016 over network 1014. Network 1014 may be any network or combination of networks of devices that communicate with one another, such as any one or any combination of a LAN (local area network), WAN (wide area network), wireless network, or other appropriate configuration.

The users of user systems 1012 may differ in their respective capacities, and the capacity of a particular user system 1012 to access information may be determined at least in part by “permissions” of the particular user system 1012. As discussed herein, permissions generally govern access to computing resources such as data objects, components, and other entities of a computing system, such as a computing platform, an application model, a social networking system, and/or a CRM database system. “Permission sets” generally refer to groups of permissions that may be assigned to users of such a computing environment. For instance, the assignments of users and permission sets may be stored in one or more databases of System 1016. Thus, users may receive permission to access certain resources. A permission server in an on-demand database service environment can store criteria data regarding the types of users and permission sets to assign to each other. For example, a computing device can provide to the server data indicating an attribute of a user (e.g., geographic location, industry, role, level of experience, etc.) and particular permissions to be assigned to the users fitting the attributes. Permission sets meeting the criteria may be selected and assigned to the users. Moreover, permissions may appear in multiple permission sets. In this way, the users can gain access to the components of a system.

In some an on-demand database service environments, an Application Programming Interface (API) may be configured to expose a collection of permissions and their assignments to users through appropriate network-based services and architectures, for instance, using Simple Object Access Protocol (SOAP) Web Service and Representational State Transfer (REST) APIs.

In some implementations, a permission set may be presented to an administrator as a container of permissions. However, each permission in such a permission set may reside in a separate API object exposed in a shared API that has a child-parent relationship with the same permission set object. This allows a given permission set to scale to millions of permissions for a user while allowing a developer to take advantage of joins across the API objects to query, insert, update, and delete any permission across the millions of possible choices. This makes the API highly scalable, reliable, and efficient for developers to use.

In some implementations, a permission set API constructed using the techniques disclosed herein can provide scalable, reliable, and efficient mechanisms for a developer to create tools that manage a user's permissions across various sets of access controls and across types of users. Administrators who use this tooling can effectively reduce their time managing a user's rights, integrate with external systems, and report on rights for auditing and troubleshooting purposes. By way of example, different users may have different capabilities with regard to accessing and modifying application and database information, depending on a user's security or permission level, also called authorization. In systems with a hierarchical role model, users at one permission level may have access to applications, data, and database information accessible by a lower permission level user, but may not have access to certain applications, database information, and data accessible by a user at a higher permission level.

As discussed above, system 1016 may provide on-demand database service to user systems 1012 using an MTS arrangement. By way of example, one tenant organization may be a company that employs a sales force where each salesperson uses system 1016 to manage their sales process. Thus, a user in such an organization may maintain contact data, leads data, customer follow-up data, performance data, goals and progress data, etc., all applicable to that user's personal sales process (e.g., in tenant data storage 1022). In this arrangement, a user may manage his or her sales efforts and cycles from a variety of devices, since relevant data and applications to interact with (e.g., access, view, modify, report, transmit, calculate, etc.) such data may be maintained and accessed by any user system 1012 having network access.

When implemented in an MTS arrangement, system 1016 may separate and share data between users and at the organization-level in a variety of manners. For example, for certain types of data each user's data might be separate from other users' data regardless of the organization employing such users. Other data may be organization-wide data, which is shared or accessible by several users or potentially all users form a given tenant organization. Thus, some data structures managed by system 1016 may be allocated at the tenant level while other data structures might be managed at the user level. Because an MTS might support multiple tenants including possible competitors, the MTS may have security protocols that keep data, applications, and application use separate. In addition to user-specific data and tenant-specific data, system 1016 may also maintain system-level data usable by multiple tenants or other data. Such system-level data may include industry reports, news, postings, and the like that are sharable between tenant organizations.

In some implementations, user systems 1012 may be client systems communicating with application servers 1050 to request and update system-level and tenant-level data from system 1016. By way of example, user systems 1012 may send one or more queries requesting data of a database maintained in tenant data storage 1022 and/or system data storage 1024. An application server 1050 of system 1016 may automatically generate one or more SQL statements (e.g., one or more SQL queries) that are designed to access the requested data. System data storage 1024 may generate query plans to access the requested data from the database.

The database systems described herein may be used for a variety of database applications. By way of example, each database can generally be viewed as a collection of objects, such as a set of logical tables, containing data fitted into predefined categories. A “table” is one representation of a data object, and may be used herein to simplify the conceptual description of objects and custom objects according to some implementations. It should be understood that “table” and “object” may be used interchangeably herein. Each table generally contains one or more data categories logically arranged as columns or fields in a viewable schema. Each row or record of a table contains an instance of data for each category defined by the fields. For example, a CRM database may include a table that describes a customer with fields for basic contact information such as name, address, phone number, fax number, etc. Another table might describe a purchase order, including fields for information such as customer, product, sale price, date, etc. In some multi-tenant database systems, standard entity tables might be provided for use by all tenants. For CRM database applications, such standard entities might include tables for case, account, contact, lead, and opportunity data objects, each containing pre-defined fields. It should be understood that the word “entity” may also be used interchangeably herein with “object” and “table”.

In some implementations, tenants may be allowed to create and store custom objects, or they may be allowed to customize standard entities or objects, for example by creating custom fields for standard objects, including custom index fields. Commonly assigned U.S. Pat. No. 7,779,039, titled CUSTOM ENTITIES AND FIELDS IN A MULTI-TENANT DATABASE SYSTEM, by Weissman et al., issued on Aug. 17, 2010, and hereby incorporated by reference in its entirety and for all purposes, teaches systems and methods for creating custom objects as well as customizing standard objects in an MTS. In certain implementations, for example, all custom entity data rows may be stored in a single multi-tenant physical table, which may contain multiple logical tables per organization. It may be transparent to customers that their multiple “tables” are in fact stored in one large table or that their data may be stored in the same table as the data of other customers.

FIG. 11A shows a system diagram of an example of architectural components of an on-demand database service environment 1100, configured in accordance with some implementations. A client machine located in the cloud 1104 may communicate with the on-demand database service environment via one or more edge routers 1108 and 1112. A client machine may include any of the examples of user systems ?12 described above. The edge routers 1108 and 1112 may communicate with one or more core switches 1120 and 1124 via firewall 1116. The core switches may communicate with a load balancer 1128, which may distribute server load over different pods, such as the pods 1140 and 1144 by communication via pod switches 1132 and 1136. The pods 1140 and 1144, which may each include one or more servers and/or other computing resources, may perform data processing and other operations used to provide on-demand services. Components of the environment may communicate with a database storage 1156 via a database firewall 1148 and a database switch 1152.

Accessing an on-demand database service environment may involve communications transmitted among a variety of different components. The environment 1100 is a simplified representation of an actual on-demand database service environment. For example, some implementations of an on-demand database service environment may include anywhere from one to many devices of each type. Additionally, an on-demand database service environment need not include each device shown, or may include additional devices not shown, in FIGS. 11A and 11B.

The cloud 1104 refers to any suitable data network or combination of data networks, which may include the Internet. Client machines located in the cloud 1104 may communicate with the on-demand database service environment 1100 to access services provided by the on-demand database service environment 1100. By way of example, client machines may access the on-demand database service environment 1100 to retrieve, store, edit, and/or process calendaring information.

In some implementations, the edge routers 1108 and 1112 route packets between the cloud 1104 and other components of the on-demand database service environment 1100. The edge routers 1108 and 1112 may employ the Border Gateway Protocol (BGP). The edge routers 1108 and 1112 may maintain a table of IP networks or ‘prefixes’, which designate network reachability among autonomous systems on the internet.

In one or more implementations, the firewall 1116 may protect the inner components of the environment 1100 from internet traffic. The firewall 1116 may block, permit, or deny access to the inner components of the on-demand database service environment 1100 based upon a set of rules and/or other criteria. The firewall 1116 may act as one or more of a packet filter, an application gateway, a stateful filter, a proxy server, or any other type of firewall.

In some implementations, the core switches 1120 and 1124 may be high-capacity switches that transfer packets within the environment 1100. The core switches 1120 and 1124 may be configured as network bridges that quickly route data between different components within the on-demand database service environment. The use of two or more core switches 1120 and 1124 may provide redundancy and/or reduced latency.

In some implementations, communication between the pods 1140 and 1144 may be conducted via the pod switches 1132 and 1136. The pod switches 1132 and 1136 may facilitate communication between the pods 1140 and 1144 and client machines, for example via core switches 1120 and 1124. Also or alternatively, the pod switches 1132 and 1136 may facilitate communication between the pods 1140 and 1144 and the database storage 1156. The load balancer 1128 may distribute workload between the pods, which may assist in improving the use of resources, increasing throughput, reducing response times, and/or reducing overhead. The load balancer 1128 may include multilayer switches to analyze and forward traffic.

In some implementations, access to the database storage 1156 may be guarded by a database firewall 1148, which may act as a computer application firewall operating at the database application layer of a protocol stack. The database firewall 1148 may protect the database storage 1156 from application attacks such as structure query language (SQL) injection, database rootkits, and unauthorized information disclosure. The database firewall 1148 may include a host using one or more forms of reverse proxy services to proxy traffic before passing it to a gateway router and/or may inspect the contents of database traffic and block certain content or database requests. The database firewall 1148 may work on the SQL application level atop the TCP/IP stack, managing applications' connection to the database or SQL management interfaces as well as intercepting and enforcing packets traveling to or from a database network or application interface.

In some implementations, the database storage 1156 may be an on-demand database system shared by many different organizations. The on-demand database service may employ a single-tenant approach, a multi-tenant approach, a virtualized approach, or any other type of database approach. Communication with the database storage 1156 may be conducted via the database switch 1152. The database storage 1156 may include various software components for handling database queries. Accordingly, the database switch 1152 may direct database queries transmitted by other components of the environment (e.g., the pods 1140 and 1144) to the correct components within the database storage 1156.

FIG. 11B shows a system diagram further illustrating an example of architectural components of an on-demand database service environment, in accordance with some implementations. The pod 1144 may be used to render services to user(s) of the on-demand database service environment 1100. The pod 1144 may include one or more content batch servers 1164, content search servers 1168, query servers 1182, file servers 1186, access control system (ACS) servers 1180, batch servers 1184, and app servers 1188. Also, the pod 1144 may include database instances 1190, quick file systems (QFS) 1192, and indexers 1194. Some or all communication between the servers in the pod 1144 may be transmitted via the switch 1136.

In some implementations, the app servers 1188 may include a framework dedicated to the execution of procedures (e.g., programs, routines, scripts) for supporting the construction of applications provided by the on-demand database service environment 1100 via the pod 1144. One or more instances of the app server 1188 may be configured to execute all or a portion of the operations of the services described herein.

In some implementations, as discussed above, the pod 1144 may include one or more database instances 1190. A database instance 1190 may be configured as an MTS in which different organizations share access to the same database, using the techniques described above. Database information may be transmitted to the indexer 1194, which may provide an index of information available in the database 1190 to file servers 1186. The QFS 1192 or other suitable filesystem may serve as a rapid-access file system for storing and accessing information available within the pod 1144. The QFS 1192 may support volume management capabilities, allowing many disks to be grouped together into a file system. The QFS 1192 may communicate with the database instances 1190, content search servers 1168 and/or indexers 1194 to identify, retrieve, move, and/or update data stored in the network file systems (NFS) 1196 and/or other storage systems.

In some implementations, one or more query servers 1182 may communicate with the NFS 1196 to retrieve and/or update information stored outside of the pod 1144. The NFS 1196 may allow servers located in the pod 1144 to access information over a network in a manner similar to how local storage is accessed. Queries from the query servers 1122 may be transmitted to the NFS 1196 via the load balancer 1128, which may distribute resource requests over various resources available in the on-demand database service environment 1100. The NFS 1196 may also communicate with the QFS 1192 to update the information stored on the NFS 1196 and/or to provide information to the QFS 1192 for use by servers located within the pod 1144.

In some implementations, the content batch servers 1164 may handle requests internal to the pod 1144. These requests may be long-running and/or not tied to a particular customer, such as requests related to log mining, cleanup work, and maintenance tasks. The content search servers 1168 may provide query and indexer functions such as functions allowing users to search through content stored in the on-demand database service environment 1100. The file servers 1186 may manage requests for information stored in the file storage 1198, which may store information such as documents, images, basic large objects (BLOBs), etc. The query servers 1182 may be used to retrieve information from one or more file systems. For example, the query system 1182 may receive requests for information from the app servers 1188 and then transmit information queries to the NFS 1196 located outside the pod 1144. The ACS servers 1180 may control access to data, hardware resources, or software resources called upon to render services provided by the pod 1144. The batch servers 1184 may process batch jobs, which are used to run tasks at specified times. Thus, the batch servers 1184 may transmit instructions to other servers, such as the app servers 1188, to trigger the batch jobs.

While some of the disclosed implementations may be described with reference to a system having an application server providing a front end for an on-demand database service capable of supporting multiple tenants, the disclosed implementations are not limited to multi-tenant databases nor deployment on application servers. Some implementations may be practiced using various database architectures such as ORACLE®, DB2® by IBM and the like without departing from the scope of present disclosure.

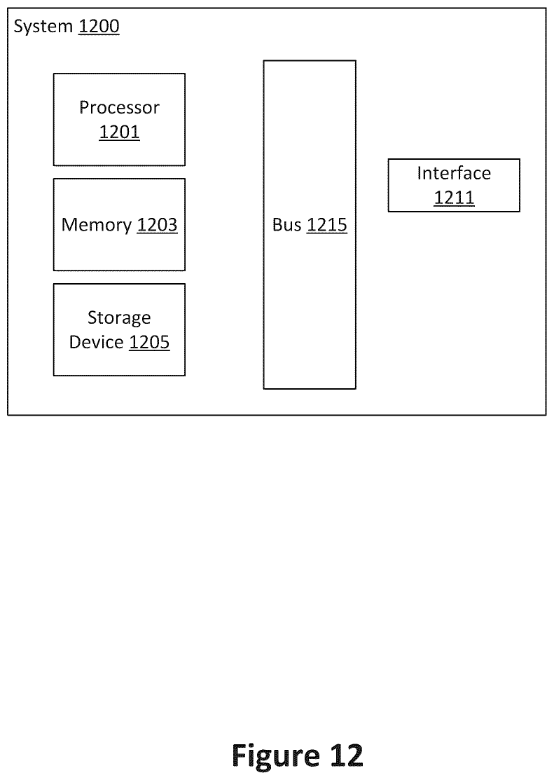

FIG. 12 illustrates one example of a computing device. According to various embodiments, a system 1200 suitable for implementing embodiments described herein includes a processor 1201, a memory module 1203, a storage device 1205, an interface 1211, and a bus 1215 (e.g., a PCI bus or other interconnection fabric.) System 1200 may operate as variety of devices such as an application server, a database server, or any other device or service described herein. Although a particular configuration is described, a variety of alternative configurations are possible. The processor 1201 may perform operations such as those described herein. Instructions for performing such operations may be embodied in the memory 1203, on one or more non-transitory computer readable media, or on some other storage device. Various specially configured devices can also be used in place of or in addition to the processor 1201. The interface 1211 may be configured to send and receive data packets over a network. Examples of supported interfaces include, but are not limited to: Ethernet, fast Ethernet, Gigabit Ethernet, frame relay, cable, digital subscriber line (DSL), token ring, Asynchronous Transfer Mode (ATM), High-Speed Serial Interface (HSSI), and Fiber Distributed Data Interface (FDDI). These interfaces may include ports appropriate for communication with the appropriate media. They may also include an independent processor and/or volatile RAM. A computer system or computing device may include or communicate with a monitor, printer, or other suitable display for providing any of the results mentioned herein to a user.

Any of the disclosed implementations may be embodied in various types of hardware, software, firmware, computer readable media, and combinations thereof. For example, some techniques disclosed herein may be implemented, at least in part, by computer-readable media that include program instructions, state information, etc., for configuring a computing system to perform various services and operations described herein. Examples of program instructions include both machine code, such as produced by a compiler, and higher-level code that may be executed via an interpreter. Instructions may be embodied in any suitable language such as, for example, Apex, Java, Python, C++, C, HTML, any other markup language, JavaScript, ActiveX, VBScript, or Perl. Examples of computer-readable media include, but are not limited to: magnetic media such as hard disks and magnetic tape; optical media such as flash memory, compact disk (CD) or digital versatile disk (DVD); magneto-optical media; and other hardware devices such as read-only memory (“ROM”) devices and random-access memory (“RAM”) devices. A computer-readable medium may be any combination of such storage devices.

In the foregoing specification, various techniques and mechanisms may have been described in singular form for clarity. However, it should be noted that some embodiments include multiple iterations of a technique or multiple instantiations of a mechanism unless otherwise noted. For example, a system uses a processor in a variety of contexts but can use multiple processors while remaining within the scope of the present disclosure unless otherwise noted. Similarly, various techniques and mechanisms may have been described as including a connection between two entities. However, a connection does not necessarily mean a direct, unimpeded connection, as a variety of other entities (e.g., bridges, controllers, gateways, etc.) may reside between the two entities.

In the foregoing specification, reference was made in detail to specific embodiments including one or more of the best modes contemplated by the inventors. While various implementations have been described herein, it should be understood that they have been presented by way of example only, and not limitation. For example, some techniques and mechanisms are described herein in the context of on-demand applications. However, the techniques disclosed herein apply to a wide variety of computing environments. Particular embodiments may be implemented without some or all of the specific details described herein. In other instances, well known process operations have not been described in detail in order to avoid unnecessarily obscuring the disclosed techniques. Accordingly, the breadth and scope of the present application should not be limited by any of the implementations described herein, but should be defined only in accordance with the claims and their equivalents.

1. A computing platform implemented using a server system, the computing platform being configurable to cause:
receiving application data from an on-demand application hosted by the computing platform;
generating a data model based, at least in part, on the application data, the data model being a calendar data structure associated with a calendaring application;
generating, using an application model, additional application data, the application model being a machine learning model; and
updating the calendar data structure of the data model based, at least in part, on the additional application data, wherein the updating is performed, at least in part, via a plurality of custom data fields of a plurality of custom data objects.

2. The system recited in claim 1, wherein the computing platform is further configurable to cause:
generating a training data set based on previous application data and historical data associated with the on-demand application; and
training the application model based on the training data set.

3. The system recited in claim 2, wherein the computing platform is further configurable to cause:
updating the training data set based on additional performance data associated with the on-demand application; and
re-training the application model based on the updated training data set.

4. The system recited in claim 1, wherein the additional application data comprises a plurality of recommended event objects.

5. The system recited in claim 4, wherein the computing platform is further configurable to cause:
generating a user interface screen configured to display the plurality of recommended event objects.

6. The system recited in claim 4, wherein the updating further comprises:
including the plurality of recommended event objects in the calendar data structure.

7. The system recited in claim 1, wherein the plurality of custom data fields comprises filtering rules, mapping rules, and operational criteria.

8. The system recited in claim 7, wherein the computing platform is further configurable to cause:
defining the plurality of filtering rules based, at least in part, on an input received from user;
defining the plurality of mapping rules to identify a plurality of syncing relationships; and
defining the operational criteria to specify one or more function calls to an additional on-demand application.

9. The system recited in claim 8, wherein the one or ore function calls are configured to trigger a process flow hosted by the additional on-demand application.

10. A method comprising:
receiving application data from an on-demand application hosted by a computing platform;
generating, using one or more processors, a data model based, at least in part, on the application data, the data model being a calendar data structure associated with a calendaring application;
generating, using an application model, additional application data, the application model being a machine learning model; and
updating, using the one or more processors, the calendar data structure of the data model based, at least in part, on the additional application data, wherein the updating is performed, at least in part, via a plurality of custom data fields of a plurality of custom data objects.

11. The method recited in claim 10 further comprising:
generating a training data set based on previous application data and historical data associated with the on-demand application; and
training the application model based on the training data set.

12. The method recited in claim 11 further comprising:
updating the training data set based on additional performance data associated with the on-demand application; and
re-training the application model based on the updated training data set.

13. The method recited in claim 10, wherein the additional application data comprises a plurality of recommended event objects, and wherein the method further comprises:
generating a user interface screen configured to display the plurality of recommended event objects.

14. The method recited in claim 13, wherein the updating further comprises:
including the plurality of recommended event objects in the calendar data structure.

15. The method recited in claim 10, wherein the plurality of custom data fields comprises filtering rules, mapping rules, and operational criteria.

16. The method recited in claim 15 further comprising:
defining the plurality of filtering rules based, at least in part, on an input received from user;
defining the plurality of mapping rules to identify a plurality of syncing relationships; and
defining the operational criteria to specify one or more function calls to an additional on-demand application.

17. One or more non-transitory computer readable media having instructions stored thereon for performing a method, the method comprising:
receiving application data from an on-demand application hosted by a computing platform;
generating a data model based, at least in part, on the application data, the data model being a calendar data structure associated with a calendaring application;
generating, using an application model, additional application data, the application model being a machine learning model; and
updating the calendar data structure of the data model based, at least in part, on the additional application data, wherein the updating is performed, at least in part, via a plurality of custom data fields of a plurality of custom data objects.

18. The one or more non-transitory computer readable media of claim 17, wherein the method further comprises:
generating a training data set based on previous application data and historical data associated with the on-demand application; and
training the application model based on the training data set.

19. The one or more non-transitory computer readable media of claim 17, wherein the plurality of custom data fields comprises filtering rules, mapping rules, and operational criteria.

20. The one or more non-transitory computer readable media of claim 19, wherein the method further comprises:
defining the plurality of filtering rules based, at least in part, on an input received from user;
defining the plurality of mapping rules to identify a plurality of syncing relationships; and
defining the operational criteria to specify one or more function calls to an additional on-demand application.
